(19) United States
(12) Patent Application Publication
(10) Pub. No.: US 2009/0220989 A1
(43) Pub. Date: Sep. 3, 2009

(54) Particle-Based Analyte Characterization

(75) Inventors: Kamala Tyagarajan, Fremont, CA (US); Dianne M. Fishwild, Mountain View, CA (US); David A. King, Menlo Park, CA (US)

Correspondence Address: BIO TECHNOLOGY LAW GROUP;C/O PORTFOLIOIP, P.O. BOX 52050, MINNEAPOLIS, MN, 55402, US

(73) Assignee: GUAVA TECHNOLOGIES, Hayward, CA (US)

(21) Appl. No.: 12/096,342

(22) PCT Filed: Dec. 5, 2006
(86) PCT No.: PCT/US2006/046661

§ 371 Date: Sep. 9, 2008

Related U.S. Application Data
(60) Provisional application No. 60/742,297, filed on Dec. 5, 2005.
(60) Provisional application No. 60/746,054, filed on May 1, 2006.

(51) Int. Cl. G01N 33/53; C12M 1/34

(52) U.S. Cl. 435/7.2; 436/501; 435/288.7

(57) ABSTRACT

Methods for assaying a sample for an analyte are provided. In various embodiments, the methods comprise contacting a sample suspected of containing the analyte with a non-uniform particle comprising a capture molecule, and further contacting the particle with a detection moiety comprising a label that permits detection of the analyte when associated with the particle. The methods may be performed to detect and/or quantitate analyte in the sample. In some embodiments, the methods may be performed in an automated manner, and may use an optical and/or cytometric apparatus for performing the method(s). The methods may further be performed with automated vessel-processing apparatus(es), such as plate loaders, plate washers, etc. Also provided are complexes containing the described materials formed by an assay of the invention, including excited state complexes. Kits useful for performing such methods are also provided.

### CROSS-REFERENCE TO RELATED APPLICATIONS

This application claims priority to U.S. provisional patent applications Ser. Nos. 60/742,297 and 60/746,054, filed Dec. 5, 2005 and May 1, 2006, respectively, each of which are incorporated herein by reference in their entirety.

### TECHNICAL FIELD

This invention relates to methods, articles and compositions relating to particle-based characterization of one or more analytes in a sample.

### BACKGROUND OF THE INVENTION

Particle-based assays in the past have often used microspheres and particles of uniform sizes and/or shapes for the evaluation of analytes in fluids of biological origin/media. Very often the size of the beads used has been dictated by the specific sample handling procedures such as washing by filtration, magnetization, and/or centrifugation.

In particular, bead sizes used for cytometry have been used in the 2 micron range or greater so a uniform population is detected (e.g. Luminex, BD, Bangs) and so they can be distinctly separated when multiplexed. Smaller particles than these sizes are difficult to utilize in procedures using wash and/or separation steps. The larger size particles currently used in detection procedures are employed where analyte concentration is low or greater sensitivity is needed, typically in the pg/mL or ng/mL range.

However, there are many instances where analytes routinely exist in much higher amounts (in the low microgram to hundreds of microgram/mL), and can only be used with existing particle-based assays with large and/or multiple dilutions, which are inconvenient and are not practical when multiple samples need to be analyzed. Additionally, in some settings, analytes may exist along with impurities that bind to the particles being used for analyte characterization and further decrease and/or modulate the range of detection due to the blocking of binding sites by impurities or interfering proteins or other molecules.

Hence the need exists for particle-based methods of analyte detection employing particles with broad and/or high capacity ranges of analyte detection. Such methods would find particular use where either a) high concentrations of analytes are being detected, b) low to medium concentrations of analytes are detected in mixtures of interfering substances such as proteins, or c) broad ranges of analyte detection are desired using a single set of assay reagents. These scenarios include, but are not limited to, detection and/or characterization of secreted proteins or other biomolecules in culture media, of analytes in cellular environments or biological fluids, and of specific analytes of interest in the presence of multiple other substances.

Thus, there is a need in the art for particle-based methods of analyzing samples for analytes, and for devices, compositions and articles of manufacture useful in such methods.

### BRIEF DESCRIPTION OF THE DRAWINGS

FIG. 1 depicts the use of a Guava® Technologies cytometry platform in the detection of an expressed cellular antigen using an analyte-specific primary antibody and a fluorescently labeled secondary antibody.

FIG. 2 is a schematic depiction of the use of a particle-based assay for detection of a representative sample analyte (an antibody in a hybridoma supernatant) using a Guava® Technologies platform.

FIG. 3 is a depiction of the process steps used in carrying out a particle-based analyte assay on a Guava® EasyCyte apparatus.

FIG. 4 provides examples of use of a particle-based assay for determining total mouse IgG on a Guava® Technologies platform.

FIG. 5A demonstrates the results from a typical calibration curve for an isotype-specific assay for a specific isotype of murine antibody using a particle-based assay on a Guava® Technologies platform. FIG. 5B shows that a panel of isotype-specific assays can be used to identify the isotype of an antibody of unknown isotype that is specific for an analyte of interest.

FIG. 6 depicts the compatibility of the methods in characterizing test analytes in various cell culture media.

FIG. 7 depicts a standard curve obtained using a method of the invention to analyze mouse IgG in test samples at known concentrations.

FIG. 8A demonstrates the correlation between concentrations determined using absorbance readings versus those obtained using a particle-based assay of the invention. FIG. 8B demonstrates that accurate concentration predictions for different murine antibody isotypes can be obtained using the methods of the invention.

FIG. 9 depicts results obtained from a murine high-capacity immunoglobulin quantitation assay provided. A different linear range was obtained by adjusting the quantities of reagents used, demonstrating that assays can be prepared for a wide variety of analyte using the methods of the invention.

FIG. 10 depicts results obtained from an assay embodiment for quantitating total human IgG in a sample. A linear range of 0.5-20 ug/ml was seen using 7.5 ul of supernatant.

FIG. 11 demonstrates the results of a universal human IgG quantitation assay in a particle-based assay format. The assay was found to accurately measure the concentrations of human antibodies of the IgG1, IgG2, IgG2 and IgG4 isotypes.

### SUMMARY OF THE INVENTION

Methods for assaying a sample for an analyte are provided. In various embodiments, the methods comprise contacting a sample suspected of containing the analyte with a non-uniform particle comprising a capture molecule, and further contacting the particle with a detection moiety comprising a label that permits detection of the analyte when associated with the particle. The methods may be performed to detect and/or quantitate analyte in the sample. In some embodiments, the methods may be performed in an automated manner, and may use an optical and/or cytometric apparatus for performing the method(s). The methods may further be performed with automated vessel-processing apparatus(es), such as plate loaders, plate washers, etc. Also provided are complexes containing the described materials formed by an assay of the invention, including excited state complexes. Kits useful for performing such methods are also provided.

### DETAILED DESCRIPTION OF THE INVENTION

The invention provides methods in which one or more analytes in a sample may be characterized using non-uniform particles comprising capture molecules specific for the analyte(s). The particles interact in a solution with a sample suspected of comprising the analyte(s). The particles are contacted with labeled detector(s) that can be localized to the particles via a binding means when the analyte is bound to the particle. Following the techniques described herein, low volumes of sample and wider concentrations of analytes can be analyzed than have previously been available in prior assays.

In some embodiments, a single assay mixture using a fluorescently-labeled detector can be applied to a cytometry platform for analysis without additional clean up steps and the fluorescent response of the particle-bound analyte can be compared to a standard curve to quantitate the analyte of interest. Higher sensitivity detection may be obtained using biotinylated detectors followed by binding to labeled streptavidin probes to amplify the signal.

In some embodiments, non-fluorescent non-uniform particles are used for binding analytes in a no-wash procedure. These particles, which now have analytes and labeled analyte-binding species bound to them, can be detected with a specific fluorescent detector. Detection can be performed on a cytometry platform (e.g., from Guava® Technologies, Hayward, Calif.) where fluorescent intensities of the particles can be obtained. Exemplary systems are described in U.S. Pats. Nos. 5,798,222, 6,710,871 and 6,816,257. Desirably, a standard curve can be used to determine the concentration of analyte(s) of interest.

The particle, sample, and detection moiety may be combined sequentially or simultaneously. Where necessary or desired, wash and/or separation steps may be incorporated at any stage of the assays described herein; conveniently, embodiments of the invention which do not require wash or separation steps are also provided, which can provide labor, reagent, cost and/or time savings.

In some embodiments, the sample is provided in at least one well or vessel of a multiwell or multivessel platform, for example using a multiwell plate. In other embodiments, the sample can be provided in a single discrete vessel, for example in a tube, a microtube or a capillary. The assay formats can be applied to any cytometry or imaging based platform.

In some embodiments, a method for analyzing a sample is provided comprising:

providing a sample suspected of comprising a first analyte, said sample comprising a fluid medium;

providing a first particle comprising or a plurality of first particles each comprising a first capture molecule for the first analyte, said first particle or each of said plurality having a non-uniform (nonspherical) shape, and wherein one, two and/or three dimensions (X-, Y- and/or Z-dimensions), or all dimensions, of the particle(s) may be less than 2 microns;

providing a first detection moiety comprising a first label, which label may be optically detectable and may be fluorescent, or may be capable of binding a substance that is optically detectable and that may be fluorescent, and a first binding means that localizes the first detection moiety to the first particle when the first analyte is bound to the capture molecule;

contacting the first particle with the sample and with the first detection moiety, sequentially or simultaneously (in some embodiments, the first particle, the sample, and the first detection moiety are combined together to create a test sample);

optionally withdrawing a test volume of fluid suspected of comprising the first particle, which may be performed automatically; and

determining whether the first label is present in the test volume or test sample (which may include determining whether the first label is associated with the first particle and to what extent), and using the information provided to determine the presence and/or concentration of the analyte(s) of interest in the sample (for example by determining whether and/or to what extent an emission associated with the first label is present in the test sample or test volume, for example by illuminating the test sample or test volume with an excitation source, and analyzing the test volume for fluorescence emission). The test volume can also be simultaneously analyzed for at least one scatter parameter, for example a scatter parameter associated with particle size and/or shape, for example forward scatter. The embodiments described herein may optionally be performed cytometrically.

In some embodiments, a method for analyzing a sample is provided comprising:

providing a sample suspected of comprising a first analyte, said sample comprising a fluid medium;

providing a first particle comprising a first capture molecule for the first analyte, said first particle having at least one diameter of less than 2 microns;

providing a first detection moiety comprising a first label and a first binding means that localizes the first detection moiety to the first particle when the first analyte is bound to the capture molecule;

contacting the first particle with the sample and with the first detection moiety; and

determining whether the first label is associated with the first particle.

In some embodiments, a method for analyzing a sample is provided comprising:

providing a sample suspected of comprising a first analyte, said sample comprising a fluid medium;

providing a first particle comprising a first capture molecule for the first analyte, said first particle having a diameter of less than 2 microns;

providing a first detection moiety comprising a first fluorescent label and a first binding means that localizes the first detection moiety to the first particle when the first analyte is bound to the capture molecule;

contacting the first particle with the sample and with the first detection moiety;

automatically withdrawing a test volume of fluid comprising the first particle;

illuminating the test volume with an excitation source;

analyzing the test volume for fluorescence emission; and

analyzing the test volume for at least one scatter parameter.

In some embodiments, a method for analyzing a sample is provided comprising:

providing a sample suspected of comprising a first analyte, said sample comprising a fluid medium;

providing a first particle comprising a first capture molecule for the first analyte, said first particle having at least one dimension of less than 2 microns and having a non-uniform shape and a higher binding capacity for the first analyte as compared to a spherical particle of the same volume;

providing a first detection moiety comprising a first label and a first binding means that localizes the first detection moiety to the first particle when the first analyte is bound to the capture molecule;

contacting the first particle with the sample and with the first detection moiety, and optionally creating a test sample by simultaneously contacting the first particle, the sample and the first detection moiety in a fluid medium; and

optionally withdrawing a test volume of fluid suspected of comprising the first particle, which may be performed automatically;

analyzing the test sample or a portion thereof or the test volume for an emission associated with the first label; and

determining the presence and/or concentration of the analyte of interest in the sample.

In the methods described herein, determining the presence and/or concentration of an analyte of interest can comprise any technique known in the art that can provide such information. In some embodiments, the methods can employ optical detection of the first label, which can provide information as to whether the first label is present at a level above background, indicating the analyte is present in the sample. In some embodiments, the amount of first label may be quantitated and used to determine the amount of analyte present in the sample. In some embodiments, determining whether the first particle is associated with the first label, and/or determining the presence and/or concentration of the analyte of interest in the sample, can comprise illuminating a test volume, test sample, solution, and/or particle with an excitation source; analyzing for a fluorescence emission upon excitation; and optionally analyzing for a scatter parameter.

In some embodiments, a method for analyzing a sample is provided comprising:

providing a sample suspected of comprising a first analyte, said sample comprising a fluid medium;

providing a first particle comprising a first capture molecule for the first analyte, said first particle having at least one dimension of less than 2 microns and having a non-uniform shape and an increased surface area as compared to a spherical particle of the same volume;

providing a first detection moiety comprising a first label and a first binding means that localizes the first detection moiety to the first particle when the first analyte is bound to the capture molecule;

contacting the first particle with the sample and with the first detection moiety, and optionally creating a test sample by simultaneously contacting the first particle, the sample and the first detection moiety in a fluid medium; and

optionally withdrawing a test volume of fluid suspected of comprising the first particle, which may be performed automatically;

analyzing the test sample or a portion thereof or the test volume for an emission associated with the first label; and

determining the presence and/or concentration of the analyte of interest in the sample.

In some embodiments, a method for analyzing a sample is provided comprising:

providing a sample suspected of comprising a first analyte and further suspected of comprising a population of cells, said sample comprising a fluid medium, said cells suspected of comprising a cellular detection moiety;

providing a first particle comprising a first capture molecule for the first analyte, said first particle having a non-uniform shape, said first particle being optically distinguishable from said cells;

providing a first detection moiety comprising a first label and a first binding means that localizes the first detection moiety to the first particle when the first analyte is bound to the capture molecule;

providing a second detection moiety comprising a second label and a second binding means that localizes the second detection moiety to the cellular detection moiety when present, wherein the first and second labels are optically distinguishable labels;

contacting the first particle with the sample and with the first detection moiety, and contacting the second detection moiety with the sample or a component thereof suspected of comprising the population of cells;

illuminating a test volume of fluid suspected of comprising the first particle and/or a cell from said population with an excitation source, which may be done by withdrawing a test volume into a defined area, for example a capillary, and may be performed automatically;

analyzing the test volume for an emission associated with one or both of the first labels; and

optionally analyzing the test volume for at least one scatter parameter.

Where a test volume is withdrawn into a capillary, the capillary may having an internal diameter sufficient to pass one particle, or cell, at a time. The test volume may be withdrawn automatically, and may be withdrawn at a uniform flow rate.

In some embodiments, a method for analyzing a sample is provided comprising:

providing a sample suspected of comprising a first analyte, said sample comprising a fluid medium;

providing a first particle comprising a first capture molecule for the first analyte;

providing a second particle comprising a second capture molecule for a second substance, wherein the second substance interferes with the assay for the first analyte, said second particle having a non-uniform shape, and the second particle is used to reduce the amount of the second substance dissolved in the sample and thereby reduce its interference with the assay for the first analyte;

providing a first detection moiety comprising a first label and a first binding means that localizes the first detection moiety to the first particle when the first analyte is bound to the capture molecule;

contacting the sample with the first particle and with the second particle;

contacting the first particle with the first detection moiety; and

determining whether the first label is associated with the first particle.

In some embodiments, a method for analyzing a sample is provided comprising:

providing a sample suspected of comprising a first analyte, said sample comprising blood or a fraction thereof;

providing a first particle comprising a first capture molecule for the first analyte;

providing a second particle comprising a second capture molecule for a blood component that interferes with the assay for the first analyte, wherein the second particle is used to reduce the amount of the second substance dissolved in the sample and thereby reduce its interference with the assay for the first analyte, said second particle having a non-uniform shape;

providing a first detection moiety comprising a fluorescent first label and a first binding means that localizes the first detection moiety to the first particle when the first analyte is bound to the capture molecule;

contacting the sample with the first particle and with the second particle;

contacting the first particle with the first detection moiety; and

automatically withdrawing a test volume of fluid comprising the first particle;

illuminating the test volume with an excitation source;

analyzing the test volume for fluorescence emission; and

analyzing the test volume for at least one scatter parameter.

In some embodiments, a method for analyzing a sample is provided comprising:

providing a sample suspected of comprising a first analyte and one or more additional substances, said sample comprising blood or a fraction thereof;

providing a first particle comprising a first capture molecule for the first analyte, said first particle having a non-uniform shape;

reducing the amount of one or more additional substances in the sample by providing a population of second particles comprising second capture molecules for at least one blood component that interferes with the assay for the first analyte;

providing a first detection moiety comprising a first label and a first binding means that localizes the first detection moiety to the first particle when the first analyte is bound to the capture molecule;

creating a test sample by means of contacting the sample, the first particle and the second particle;

contacting the test sample with the first detection moiety;

automatically analyzing the test sample for an emission associated with the first label; and

determining the presence and/or concentration of the first analyte in the sample.

Any of the specific embodiments described herein may be modified as described for the variations in the particular method steps. Further variations of the embodiments are described herein.

In various embodiments, aspects of the invention include the use of using smaller size beads or microsphere particles which may include a uniform or non-uniform mixture of particles in bead based assays using cytometry platforms with fluorimetry or other optical detection methods. Such particle sets can comprise uniformly or non-uniformly shaped particles.

Such particles provide the advantage of increased surface area for reaction and increased capacity of binding. Further, the smaller size of these particles permits their retention in solution for a longer period of time, which promotes better contact and reactivity with analytes. The better suspension properties of these beads can allow the use of automated sample preparation stations, and may be used without vigorous shaking and/or wash steps.

The smaller size of the particles provides the added advantage that they may have optically distinguishable characteristics, for example exhibiting much lower forward scatter. Hence they can be optically (and/or physically) separated from cells on basis of scatter characteristics. Cells can exhibit optically distinguishable characteristics, such as scatter parameter(s) or the presence of detectable moieties, that can permit different types and subpopulations of cells to be distinguished, as well as permitting their optical distinction from different particles or set of distinguishable particles. For example, the forward scatter characteristics of small particles are well separated from eukaryotic cells. Particles can therefore be used in experiments where both cell and bead populations in the same sample need to be analyzed, and may be used in formats employing multiplexing of different particles and/or cells.

Higher capacity particle sets as described herein can be used in settings where a high concentration of impurities exists and the high capacity particles can be used to bind the impurities thereby reducing background signal and permitting analytes at lower concentrations to be better detected. This can be accomplished in a purely particle-based format by using particles with distinguishable optical characteristics (e.g., larger forward scatter parameters) to detect the analyte of interest after using high capacity particles to reduce or eliminate the free impurities in the assay medium.

The invention has particular application in research and development screening, production and manufacturing scenarios where characterization of analytes in the nanogram/mL to microgram/mL range is typically required. Use of particles in the provided methods can permit use of a single assay format for characterization of analytes at a variety of concentrations as may occur in different assays used at various stages of product development.

Multiplex methods are provided for assaying 2, 3, 4, 5, 10 15, 20, 25, 50, 100, 200, 500, 1000 or more different analytes and/or cells using different particles and/or cellular detection moieties, and can be used simultaneously, in parallel, or sequentially, employing different optionally encoded particles, detection moieties, and/or labels, in various permutations. The particles can be encoded internally and/or externally (e.g. using dyes) to permit multiplexing using distinguishable particles in certain multiplexing formats. Multiplexing may also be achieved by using different size particles, adding an additional parameter that can be varied to multiply the number of analytes that can be tested using a defined dye set. For example, different size particles can incorporate different analyte-binding species, which then could be detected using an identically labeled secondary binding molecule. Detection of the same label in association with particles of a known size would then provide identification (and quantitation if desired) of a particular analyte.

Multiple different types of assays may also be performed in parallel from the same sample, including without limitation detection assays, characterization assays, quantitation assays, and functional assays regarding bioproperties or other parameters exhibited by or reflected in the analytes (e.g., ADCC, complement binding, blocking studies, epitope mapping, affinity measurements, etc.).

Also provided are complexes produced by such methods, said complexes comprising a particle, an analyte, and a detection moiety. These complexes include excited state complexes produced by illuminating a complex with an excitation source that can excite a suitable label. Kits comprising components useful the methods are also provided.

The inventions described herein can be used for any assay in which a sample is interrogated regarding an analyte. Typical assays might involve determining the presence of the analyte in the sample, the relative amount of the analyte, or may be quantitative or semi-quantitative regarding the amount of analyte in the sample. For example, cells may be subjected to different stimuli, and samples prepared from such cells and/or from their culture medium may be tested to determine the effect of those stimuli using the methods of the invention.

Before the present invention is further described, it is to be understood that this invention is not limited to the particular methodology, devices, solutions or apparatuses described, as such methods, devices, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “an analyte” includes a plurality of analytes, reference to “a particle” includes a plurality of such particles, reference to “a sample” includes a plurality of samples, and the like.

Terms such as “connected,” “attached,” “linked,” and the like are used interchangeably herein and encompass direct as well as indirect connection, attachment, or linkage unless the context clearly dictates otherwise, and includes chemical couplings as well as nonchemical binding or other association. Thus, these terms intend that the particles, chemicals, labels, etc., which are “linked” may be physically linked by, for example, covalent chemical bonds, physical forces such van der Waals or hydrophobic interactions, encapsulation, embedding, or the like. For example, detection moieties can be associated with a biotin label which can bind to a corresponding biotin-binding species (e.g., avidin or streptavidin or modified forms).

Where a range of values is recited, it is to be understood that each intervening integer value, and each fraction thereof, between the recited upper and lower limits of that range is also specifically disclosed. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. For any element of an invention for which a plurality of options are disclosed, examples of that invention in which each of those options is individually excluded along with all possible combinations of excluded options are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

### DEFINITIONS

In describing the present invention, the following terms will be employed, and are intended to be defined as indicated below.

The terms “polynucleotide,” “oligonucleotide,” “nucleic acid” and “nucleic acid molecule” are used herein to include a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms 4“polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide. Some or all of the residues in the polynucleotide can optionally be modified in one or more ways.

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al., (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other nonnatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione. Other such modified nucleotidic units which form unique base pairs are known, such as those described in Leach et al. (1992) J. Am. Chem. Soc. 114:3675-3683 and Switzer et al., supra.

“Nucleic acid probe” and “probe” are used interchangeably and refer to a structure comprising a polynucleotide, as defined above, that contains a nucleic acid sequence that can bind to a corresponding analyte. The polynucleotide regions of probes may be composed of DNA, and/or RNA, and/or synthetic nucleotide analogs.

“Complementary” or “substantially complementary” refers to the hybridization or base pairing between nucleotides or nucleic acids, such as, for instance, between the two strands of a double stranded DNA molecule or between a polynucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984).

Stringent hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone.

The terms “aptamer” (or “nucleic acid antibody”) is used herein to refer to a single- or double-stranded polynucleotide that recognizes and binds to a molecule of interest by virtue of its shape. See, e.g., PCT Publication Nos. WO 92/14843, WO 91/19813, and WO 92/05285.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides containing [post-translational] modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or esterifications of carboxyl groups) and the like are included within the meaning of polypeptide. By “modified” with reference to proteins (including antibodies), and other biomolecules, is meant a modification in one or more functional groups, for example any portion of an amino acid, the structure and/or location of a sugar or other carbohydrate, or other substituents of biomolecules, and can include without limitation chemical modifications (e.g., succinylation, acylation, the structure and/or location of disulfide bonds), as well as noncovalent binding (e.g., of a small molecule, including a drug).

As used herein, the term “binding pair” refers to first and second molecules that bind specifically to each other with greater affinity than to other components in the sample. The binding between the members of the binding pair is typically noncovalent. Exemplary binding pairs include immunological binding pairs (e.g. any haptenic or antigenic compound in combination with a corresponding antibody or binding portion or fragment thereof, for example digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin), IgG and protein A, IgG and protein G, IgG and protein L, and nonimmunological binding pairs (e.g., biotin and a biotin binding substance [including avidin, streptavidin, or a derivative of either thereof], nucleotides and nucleotide-binding proteins, hormone [e.g., thyroxine and cortisol]-hormone binding protein, receptor-receptor agonist or antagonist (e.g., acetylcholine receptor-acetylcholine or an analog thereof) IgG-protein A, lectin-carbohydrate, enzyme-enzyme cofactor, enzyme-enzyme-inhibitor, an organic or inorganic molecule and a biomolecule that binds to the molecule, and two polynucleotides capable of forming nucleic acid duplexes and/or higher order structures) and the like. One or both members of the binding pair can be conjugated to additional molecules.

The term “antibody” as used herein includes antibodies obtained from both polyclonal and monoclonal preparations, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expression human immunoglobulin chain genes or portions thereof. See, e.g., Cote, et al. Monoclonal Antibodies and Cancer Therapy, Alan R. Liss, 1985, p. 77.

The terms “semiconductor nanocrystal,” “SCNC,” and “quantum dot” are used interchangeably herein and refer to an inorganic crystallite of about 1 nm or more and about 1000 nm or less in diameter or any integer or fraction of an integer therebetween, preferably at least about 2 nm and about 50 nm or less in diameter or any integer or fraction of an integer therebetween, more preferably at least about 2 nm and about 20 nm or less in diameter (for example about 2, 3, 4, 5, 6, 7, 8, 9, 10, 11, 12, 13, 14, 15, 16, 17, 18, 19, or 20 nm). SCNCs are characterized by their uniform nanometer size. An SCNC is capable of emitting electromagnetic radiation upon excitation (i.e., the SCNC is luminescent) and includes a “core” of one or more first semiconductor materials, and may be surrounded by a “shell” of a second semiconductor material. An SCNC core surrounded by a semiconductor shell is referred to as a “core/shell” SCNC. The surrounding “shell” material will preferably have a bandgap energy that is larger than the bandgap energy of the core material and may be chosen to have an atomic spacing close to that of the “core” substrate. The core and/or the shell can be a semiconductor material including, but not limited to, those of the group II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like) materials, and an alloy or a mixture thereof. Thus, the terms “semiconductor nanocrystal,” “SCNC,” and “quantum dot” as used herein include a coated SCNC core, as well as a core/shell SCNC.

“Multiplexing” herein refers to an assay or other analytical method in which multiple analytes can be assayed simultaneously.

“Optional” or “optionally” means that the subsequently described event or circumstance may or may not occur, and that the description includes instances where the event or circumstance occurs and instances in which it does not.

### The Sample and Analyte

In principle, the sample can be any material suspected of containing an analyte of interest, and is typically provided in or dissolved or dispersed in a fluid medium. The analyte may be a biomolecule, for example a peptide or protein, a polynucleotide such as DNA or RNA, an antibody, saccharides, oligosaccharides, polysaccharides, etc. The analyte may be a small molecule, and may be organic or inorganic.

In some embodiments, the sample or portion of the sample comprising or suspected of comprising the analyte can be any source of biological material, including cells, tissue or fluid, including bodily fluids, and the deposits left by that organism, including viruses, mycoplasma, and fossils. Typically, the sample is obtained as or dispersed in a predominantly aqueous medium. Nonlimiting examples of the sample include blood, urine, semen, milk, sputum, mucus, a buccal swab, a lavage, a vaginal swab, a rectal swab, an aspirate, a needle biopsy, a section of tissue obtained for example by surgery or autopsy, plasma, serum, spinal fluid, cerebrospinal fluid, amniotic fluid, lymph fluid, the external secretions of the skin, respiratory, intestinal, and genitourinary tracts, tears, saliva, tumors, organs, samples of in vitro cell culture constituents (including but not limited to conditioned medium resulting from the growth of cells in cell culture medium, putatively virally infected cells, recombinant cells, and cell components, including without limitation hybridoma supernatants producing human or murine antibodies and supernatants from cells producing fragments or modified forms of antibodies or other immunological or secreted proteins), a cellular lysate, and a recombinant library comprising polynucleotide sequences.

The sample can be a positive control sample which is known to contain the analyte. A negative control sample can also be used which, although not expected to contain the analyte is suspected of containing it, and is tested in order to confirm the lack of contamination by the analyte of the reagents used in a given assay, as well as to determine whether a given set of assay conditions produces false positives (a positive signal even in the absence of analyte in the sample).

The sample can be diluted, dissolved, suspended, purified, extracted or otherwise treated to solubilize or resuspend any analyte present or to render it accessible to reagents.

### The Particles

The particles used in the described methods are non-uniform/irregular in shape. The particles may have at least two different (X-, Y- and/or Z-) dimensions, and may have three (or more, for unusually shaped particles) different dimensions. The particles are therefore nonspherical, having a shape other than that of a solid sphere. In some embodiments, the particles exhibit an increased surface area over a sphere or other solid shape occupying the same volume. Desirably, the non-uniform particles exhibit an irregular surface (on a macro- and/or micro-scale) that produces a large increase in surface area. The particles desirably exhibit at least a two-fold increase in surface area, and may exhibit at least a three-fold, five-fold, 10-fold or 20-fold increase in surface area. The particles may exhibit up to a 30-fold, 40-fold, 50-fold, 100-fold, or 200-fold increase in surface area over a similarly sized smooth spherical particle. The particles may exhibit an increased binding capacity over a similarly-sized spherical particle, which may result from the increased surface area and/or from an increase in the density of capture moieties (or derivatizable functionalities) used to bind analyte.

Desirably, at least one, two or three (or all) dimensions of the particle may be less than about 30 or 40 microns, as is compatible with flow cytometric systems, and may be less than about 20 microns, less than about 10 microns, or less than about 2 microns in such dimensions. With reference to these dimensions, it is understood that such particles are typically provided as distributions of different sizes, and that particles will exhibit mean distributions meeting this limitation, such that an average particle in a population will meet such limitation(s).

The particles can be used for the detection and/or quantitation of any analyte that can be bound by a capture molecule and detected using a detection moiety, such as biomolecules, including proteins, peptides, oligonucleotides, and carbohydrates, as well as small molecule analytes. The use of smaller, higher capacity particles than have previously been used can represent considerable cost savings for the design of simple analyte assays, including immunoassays, on cytometry platforms.

In some embodiments, the particles are optically distinguishable from other substances used in the assay, for example cells and/or one or more other populations of particles, for example by a scatter parameter such as forward scatter. The particles may be generally bead-like, although lacking a uniform spherical surface, and may be porous, microporous or macroporous, or may be nonporous. Particles having a mean diameter of less than 2 microns may be desirable, as they can exhibit improved suspension properties which can lead to increased contact with the test sample and/or higher binding capacities. The particles can be obtained or derivatized to comprise a capture molecule for the analyte of interest.

The particle may be formed from any material(s) which are compatible with the methods of the invention. The particle can comprise a wide range of material, including organic materials, inorganic materials, or a combination of any of these. For example, the particle may comprise a polymerized Langmuir Blodgett film, functionalized glass, carbon, metal(s), plastics, resins, inorganic glasses, Si, Ge, GaAs, GaP, SiO₂, SiN₄, modified silicon, or any one of a wide variety of gels or polymers, including (poly)tetrafluoroethylene, (poly)vinylidenedifluoride, polyethylene, polypropylene, polyvinylchloride, a polyamide (e.g., Nylon), a polyurethane, polyvinylpyrrolidone, a polyvinyl alcohol, polyvinylacetate, cellulose acetate, polystyrene, polytetrafluoroethylene, a polyester (e.g. polyethylene terphthalate (Dacron)), cross-linked polystyrene, polyacrylic, polylactic acid, polyglycolic acid, poly(lactide coglycolide), polyanhydrides, poly(methyl methacrylate), poly(ethylene-co-vinyl acetate), polysaccharides, polysiloxanes, polymeric silica, latexes, dextran polymers, epoxies, polycarbonate, or combinations thereof. The particle may comprise a material selected from the group consisting of a metal oxide, a silicate, and a polymer, and a combination thereof. The particles may comprise a material selected from the group consisting of an iron-oxide, silica, polystyrene, polyacrylate, polymethylmethacrylate, and polydivinylbenzene. The particle may comprise a material that is magnetic, paramagnetic, superparamagnetic or non-magnetic. The particles are typically provided in plurality for use in the methods of the invention.

The particles may prepared with a size distribution of interest, or may be modified to obtain the desired parameters. For example, particles with desired properties may be obtained by suspension polymerization, or may be obtained by bulk polymerization which are then ground to produce smaller particles. Where the initial production does not produce particles with desired size distributions, such particles may be obtained through sieving or other separation techniques. Any available technique which produces particles useful in the invention may be used. Exemplary sources of particles include Bangs Labs, Spherotech, Dynal and Polysciences.

Capture molecules can be fabricated on or attached to the particle by any available method; suitable methods are known in the art, including a variety of coupling chemistries. The particles may be prepared or derivatized to comprise surface functionalities which can be coupled to suitable functionalities incorporated into the capture molecules. Examples of methods for synthesizing capture molecules on particles include those described in U.S. Pat. No. 5,143,854, PCT WO Pub. No. 92/10092, U.S. patent application Ser. No. 07/624,120, filed Dec. 6, 1990 (now abandoned), Fodor et al., Science, 251: 767-777 (1991), and PCT Pub. No. WO 90/15070. In some instances, particles containing capture molecules are commercially available.

The capture molecule can, of course, bind to the analyte of interest, and is typically one member of a binding pair. In some embodiments, the capture molecule is one member of a binding pair selected from the group consisting of an immunological binding pair, biotin and a biotin binding substance, a hormone and a hormone binding protein, a receptor and a receptor agonist or antagonist, IgG and protein A, IgG and protein G, IgG and protein L, antigen and antibody, a polynucleotide and a polynucleotide-binding protein, a lectin and a carbohydrate, an enzyme and an enzyme cofactor, an enzyme and an enzyme inhibitor; an organic or inorganic molecule and a biomolecule that binds to the molecule, and two polynucleotides capable of forming a nucleic acid duplex or multiplex.

### The Detection Moiety

A detection moiety comprising a binding means specific for an analyte when bound to a particle is used in the assays provided. A label is attached to the detection moiety in order for the capture of the analyte(s) to be more easily detected. In certain multiplex formats, the labels used for detecting different analytes may be distinguishable. The label is conjugated, directly or indirectly, to the detection moiety. Many labels are commercially available in activated forms which can readily be used for such conjugation (for example through amine acylation), or labels may be attached through known or determinable conjugation schemes many of which are well-characterized in the art.

The binding means is thus also one member of a binding pair, and may be a member selected from the group consisting of an immunological binding pair, biotin and a biotin binding substance, a hormone and a hormone binding protein, a receptor and a receptor agonist or antagonist, IgG and protein A, IgG and protein G, IgG and protein L, antigen and antibody, a polynucleotide and a polynucleotide binding protein, a lectin and a carbohydrate, an enzyme and an enzyme cofactor, an enzyme and an enzyme inhibitor, an organic or inorganic molecule and a biomolecule that binds to the molecule, and two polynucleotides capable of forming a nucleic acid duplex or multiplex.

The label which is ultimately detected may be bound through a variety of intermediate linkages. For example, a detection moiety may comprise a biotin-binding species, and an optically detectable label may be conjugated to biotin and then bound to a particle-bound detection moiety where the analyte is present and bound to the particle. Similarly, the detection moiety may comprise an immunological species such as an antibody or fragment, and a secondary antibody containing an optically detectable label may be added and localized to a particle-bound analyte. Similar schemes can be envisioned, and all such embodiments comprising a binding means specific for one or more particle-bound analytes and a detectable label, in whatever variations, that permit an assay for an analyte are useful as detections moieties.

Labels useful in the invention described herein include any substance which can be detected in association with the particle when the detection moiety to which the label is attached is bound to the analyte. Any effective detection method can be used, including optical, spectroscopic, electrical, piezoelectrical, magnetic, Raman scattering, surface plasmon resonance, calorimetric, calorimetric, etc.

The label typically comprises an agent selected from chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may also be used, wherein a quencher and a fluorophore may be used on the detection moiety and the particle(s) and/or cell(s), such that a change in optical parameters of the particle(s) and/or cell(s) occurs upon binding of the detection moiety such that a signal may be introduced or quenched a signal from the fluorophore; thus the label may be one member of a quenching pair. Suitable quencher/fluorophore systems are known in the art.

Where the label is a chromogen, the chromogen may be fluorescent or luminescent, including the fluorescent chromogens described in U.S. Pat. No. 5,912,139, as well as some tetrazolium salts. The chromogen may undergo a visually detectable change, for example from colorless or nearly colorless to a deep color, which change may require an additional method step to accomplish. For quantitative assays in solution or for assays using light absorbance in the detection method, for example in a multiwell tray setting, soluble reaction products are preferred so as to avoid errors introduced by the scattering of light from deposited insoluble products.

Exemplary chromogens include methyl blue, 2,6-dichlorophenolindophenol, resazurin, Fe^{III}-phenanthroline complex, alamar blue, the thiol-responsive indicator dyes described in U.S. Pat. No. 5,510,245, and tetrazolium salts. The chromogen is used in an amount that produces a detectable signal upon its conversion by the hydride abstractor in the presence of reduced cofactor, and can be empirically determined for a given assay system; typical amounts of chromogen range from about 1 μg to about 500 mg for small scale assays.

Exemplary tetrazolium salts that can be used or tested for their applicability as chromogens in a particular embodiment of the invention include: nitroblue tetrazolium chloride (NBT, 2H-(Tetrazolium,-3,3′-(3,3′-dimethoxy(1,1′-biphenyl)-4,4′-diyl)bis(4-nitrophenyl)-5-(phenyl), dichloride); 3-(4,5-Dimethyl-2-thiazolyl)-2,5-diphenyl-2H-tetrazolium bromide (MTT; thiazolyl blue); iodonitrotetrazolium chloride (INT; 2-(4-Iodophenyl)-3-(4-nitrophenyl)-5-phenyl-2H-tetrazolium chloride; iodonitrotetrazolium violet); 3-(4-Iodophenyl)-2-(4-nitrophenyl)-5-phenyl-2H-tetrazolium chloride; neotetrazolium chloride (NTC; 2,2′,5,5′-Tetraphenyl-3,3′-[p-diphenylene]ditetrazolium chloride); tetranitro tetrazolium blue chloride (TNBT; 2,2′,5,5′-Tetra(4-nitrophenyl)-3,3′-dimethoxy-4,4′-biphenylene)-2H,2H′-ditetrazolium chloride); tetrazolium Blue chloride (BT; blue tetrazolium chloride; 2,2′,5,5′-Tetraphenyl-3,3′-(3,3′-di methoxy-4,4′-biphenylene)-2H,2H′-ditetrazolium chloride); triphenyltetrazolium chloride (TTC; tetrazolium red; 2,3,5-Triphenyl-2H-tetrazolium chloride); triphenyltetrazolium bromide (TTB; 2,3,5-Triphenyl-2H-tetrazolium bromide); 4-[3-(4-Iodophenyl)-2-(4-nitrophenyl)-2H-5-tetrazolio]-1,3-benzene disulfonate (WST 1); 4-[3-(4-Iodophenyl)-2-(2,4-dinitrophenyl)-2H-5-tetrazolio]-1,3-benzenedisulfonate (WST 3); 2-Benzothiazolyl-3-(4-carboxy-2-methoxyphenyl)-5-[4-(2-sulfoethylcarbamoyl)phenyl]-2H-tetrazolium salt (WST 4); 2,2′-dibenzothiazolyl-5,5′-bis(4-di(2-sulfoethyl)carbamoylphenyl)-3,3′-(3,3′-dimethoxy-4,4′-biphenylene)ditetrazolium, disodium salt (WST-5); Sodium 3,3′-{1-[(Phenylamino)carbonyl]-3,4-tetrazolium}-bis(4-methoxy-6-nitro)benzenesulfonic acid hydrate (XTT); 2-(2′-benzothiazolyl)-5-styryl-3-(4′-phthalhydrazidyl)tetrazolium (BSPT); 2-benzothiazolyl-(2)-3,5-diphenyl tetrazolium (BTDP); 2,3-di(4-nitrophenyl)tetrazolium (DNP); 2,5-diphenyl-3-(4-styrylphenyl)tetrazolium (DPSP); distyryl nitroblue tetrazolium (DS-NBT); 2-phenyl-3-(4-carboxyphenyl)-5-methyl tetrazolium (PCPM); thiocarbamyl nitroblue tetrazolium (TCNBT; 2,2′-Di(p-nitrophenyl)-5,5′-di(p-thiocarbamylphenyl)-3,3′-(3,3′-dimethoxy-4,4′-biphenylene)ditetrazolium chloride); 5-cyano-2,3-di-4-tolyl-tetrazolium chloride (CTC); Nitrotetrazolium Violet (NTV); p-Anisyl Blue Tetrazolium Chloride (pABT); m-Nitro Neotetrazolium Chloride (m-NNT); o-Tolyl Tetrazolium Red (o-TTR); p-Tolyl Tetrazolium Red (pTTR); Piperonyl Tetrazolium Blue (PTB); p-Anisyl-p-Nitro Blue Tetrazolium Chloride (pApNBT); Veratryl Tetrazolium Blue (VTB); and tetrazolium violet (TV; 2,5-Diphenyl-3-(alpha-naphthyl)tetrazolium chloride), all of which are commercially available (e.g., Fluka, Calbiochem, Serva, Sigma-Aldrich, Amersham Biosciences, Connect Marketing GmbH (Buchs, Switzerland)) and/or can be synthesized via published techniques.

Chromophores useful in the methods described herein include any substance which can absorb energy and emit light. Chemical methods for attaching a signaling chromophore to a sensor molecule or other assay component are known. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Exemplary fluorescent dyes include fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2®, Cy3®, Cy3.5®, Cy5®, Cy5.5®, Cy-Chrome, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), APC-Cy5, APC-Cy7, phycoerythrin (PE), PECy5, PECy7,7-amino-4-methylcoumarin-3-acetic acid, BODIPY® FL, BODIPY® FL-Br₂, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, conjugates thereof, and combinations thereof. Exemplary lanthanide chelates include europium chelates, terbium chelates and samarium chelates.

Other dyes and fluorophores are described at www.probes.com (Molecular Probes, Inc.).

The term “green fluorescent protein” refers to both native Aequorea green fluorescent protein and mutated versions that have been identified as exhibiting altered fluorescence characteristics, including altered excitation and emission maxima, as well as excitation and emission spectra of different shapes (Delagrave, S. et al. (1995) Bio/Technology 13:151-154; Heim, R. et al. (1994) Proc. Natl. Acad. Sci. USA 91:12501-12504; Heim, R. et al. (1995) Nature 373:663-664). Delagrave et al. isolated mutants of cloned Aequorea Victoria GFP that had red-shifted excitation spectra. Heim, R. et al. reported a mutant (Tyr66 to His) having a blue fluorescence.

A wide variety of fluorescent semiconductor nanocrystals (“SCNCs”) are known in the art; methods of producing and utilizing semiconductor nanocrystals are described in: PCT Publ. No. WO 99126299 published May 27, 1999, inventors Bawendi et al.; U.S. Pat. No. 5,990,479 issued Nov. 23, 1999 to Weiss et al.; and Bruchez et al., Science 281:2013, 1998. Semiconductor nanocrystals can be obtained with very narrow emission bands with well-defined peak emission wavelengths, allowing for a large number of different SCNCs to be used as signaling chromophores in the same assay, optionally in combination with other non-SCNC types of signaling chromophores. SCNCs for use in the subject methods can be made from any material and by any technique that produces SCNCs having emission characteristics useful in the methods, articles and compositions taught herein. Exemplary methods of production are disclosed in U.S. Pats. Nos. 6,048,616; 5,990,479; 5,690,807; 5,505,928; 5,262,357, as well as PCT Publication No. WO 99/26299 (published May 27, 1999).

The SCNCs have absorption and emission spectra that depend on their size, size distribution and composition. These SCNCs can be prepared as described in Murray et al. (1993) J. Am. Chem. Soc. 115:8706-8715, Guzelian et al. (1996) J. Phys. Chem. 100:7212-7219 or PCT Publ. No. WO 99/26299 (inventors Bawendi et al.).

Examples of materials from which SCNCs can be formed include group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge, Si, and ternary and quaternary mixtures thereof. Exemplary SCNCs that emit energy in the visible range include CdS, CdSe, CdTe, ZnSe, ZnTe, GaP, and GaAs. Exemplary SCNCs that emit energy in the near IR range include InP, InAs, InSb, PbS, and PbSe. Exemplary SCNCs that emit energy in the blue to near-ultraviolet include ZnS and GaN. The size of SCNCs in a given population can be determined by the synthetic scheme used and/or through use of separation schemes, including for example size-selective precipitation and/or centrifugation. The separation schemes can be employed at an intermediate step in the synthetic scheme or after synthesis has been completed. For a given composition, larger SCNCs absorb and emit light at longer wavelengths than smaller SCNCs. SCNCs absorb strongly in the visible and UV and can be excited efficiently at wavelengths shorter than their emission peak. This characteristic allows the use in a mixed population of SCNCs of a single excitation source to excite all the SCNCs if the source has a shorter wavelength than the shortest SCNC emission wavelength within the mixture; it also confers the ability to selectively excite subpopulation(s) of SCNCs within the mixture by judicious choice of excitation wavelength.

The surface of the SCNC is preferably modified to enhance emission efficiency by adding an overcoating layer to form a “shell” around the “core” SCNC, because defects in the surface of the core SCNC can trap electrons or holes and degrade its electrical and optical properties. Addition of an insulating shell layer eliminates nonradiative relaxation pathways from the excited core, resulting in higher luminescence efficiency. Suitable materials for the shell include semiconductor materials having a higher bandgap energy than the core and preferably also having good conductance and valence band offset. Thus, the conductance band of the shell is desirably of a higher energy and the valence band is desirably of a lower energy than those of the core. For SCNC cores that emit energy in the visible (e.g., CdS, CdSe, CdTe, ZnSe, ZnTe, GaP, GaAs) or near IR (e.g., InP, InAs, InSb, PbS, PbSe), a material that has a bandgap energy in the ultraviolet may be used for the shell, for example ZnS, GaN, and magnesium chalcogenides, e.g., MgS, MgSe, and MgTe. For an SCNC core that emits in the near IR, materials having a bandgap energy in the visible, such as CdS or CdSe, or the ultraviolet may be used. Preparation of core-shell SCNCs is described in, e.g., Dabbousi et al. (1997) J. Phys. Chem. B 101:9463; Kuno et al., J. Phys. Chem. 106:9869 (1997); Hines et al., J. Phys. Chem. 100:468; and PCT Publ. No. WO 99/26299. The SCNCs can be made further luminescent through overcoating procedures as described in Danek et al. (1996) Chem. Mat. 8(1):173-180, Peng et al. (1997) J. Am. Chem. Soc. 119:7019-7029.

### Excitation and Detection

Any instrument that provides a wavelength that can excite the label and is shorter than the emission wavelength(s) to be detected can be used for excitation. Commercially available devices can provide suitable excitation wavelengths as well as suitable detection components. Any electromagnetic emission wavelength that can be produced and detected can be used, including visible wavelengths, ultraviolet wavelengths, and infrared wavelengths.

Exemplary excitation sources include a broadband UV light source such as a deuterium lamp with an appropriate filter, the output of a white light source such as a xenon lamp or a deuterium lamp after passing through a monochromator to extract out the desired wavelengths, a continuous wave (cw) gas laser, a solid state diode laser, or any of the pulsed lasers. Emitted light can be detected through any suitable device or technique; many suitable approaches are known in the art.

Incident light wavelengths useful for excitation can include 300 nm to 1000 nm wavelength light. Exemplary useful incident light wavelengths include, but are not limited to, wavelengths of at least about 300, 350, 400, 450, 500, 550, 600, 700, 800 or 900 nm, and may be less than about 1000, 900, 800, 700, 600, 550 or 500 nm. Exemplary useful incident light in the region of 450 nm to 500 nm, 500 nm to 550 nm, 550 nm to 600 nm, 600 nm to 700 nm, and 700 nm to 1000 nm. In certain embodiments, the complexes form an excited state upon illumination with incident light having a wavelength including a wavelength of about 488 nm, about 532 nm, about 594 nm and/or about 633 nm. Additionally, useful incident light wavelengths can include, but are not limited to, 488 nm, 532 nm, 594 nm and 633 nm wavelength light.

Any apparatus that can detect a label used on a detection moiety for analyte characterization when bound to a particle may be used, including without limitation flow cytometers, which may be hydrodynamically focused, imaging systems, imaging flow cytometers, and plate-based imaging systems. Nonlimiting examples of systems useful with the present methods include the Guava® EasyCyte™, the Guava® EasyCyte™ Mini, the Guava® PCA™, the Guava® PCA™-96, the Guava® EasyCyte™ Plus, FACS™ Aria, FACS™ Canto, Beckman Coulter Quanta™, Amnis ImageStream™, Molecular Devices ImageXpress™ apparatuses, and similar devices. Other apparatuses, including plate loading, plate washing, plate rocking, and similar devices useful for handling any components of the assays described herein may be used in conjunction with an apparatus used to perform the particle-based assay.

### Kits

Kits comprising reagents useful for performing the methods of the invention are also provided. In some embodiments, a kit comprises:

a first vessel containing a population of first particles, each of said population comprising a plurality of first capture molecules for a first analyte; the particles may be any of those embodiments set forth above, and may have at least one dimension of less than 2 microns;

a second vessel containing a plurality of first detection moieties, each of said plurality comprising an optically detectable first label and a first binding means capable of localizing to the first particle when the first analyte is bound to the capture molecule; and

(a) a housing for retaining the vessels, or

(b) instructions for use of the components of the kit, or

(c) both (a) and (b).

The kit may comprise at least one vessel containing a standard for calibrating the concentration of the first analyte, and may also comprise vessel(s) for standard(s) for additional analytes. The kit may also comprise a fourth vessel containing a buffer solution for performing the assay. The kit may also comprise a fifth vessel containing a buffer solution used to dilute the sample after a final incubation step and prior to data acquisition.

The kit may also support the use of a variety of multiplex formats. In some embodiments, the kit may comprise a plurality of vessels each containing a different population of particles as described herein, each of said different populations comprising capture molecules specific for a different analyte. The kit may comprise a plurality of vessels each containing a different plurality of detection moieties, each of said different plurality of detection moieties comprising binding means specific for a different analyte. The kit may comprise one or more different capture molecules and/or optically detectable labels.

The kit may also comprise one or more standards for calibrating the concentration of the first analyte, and may also comprise standards for calibrating the concentration of other analyte(s).

Various components of the kit may be provided in solution, or may require addition of a fluid medium prior to use in the assay. Kit components may independently be provided at concentrations ready for use in the assay, or may be provided at other concentrations which must be altered prior to assay, for example by dilution. One or more additional solutions may be provided in the kits, including without limitation buffer solution(s) in which the assay may be performed and/or various kit components (and/or the sample) may be diluted or dissolved. A buffer used to dilute the sample after a final incubation step and prior to data acquisition may be provided in the kit.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Materials and Methods

### Materials:

All reagents were obtained from Guava™ Technologies or are otherwise described herein. Standard Hybridoma Media used was 90% ATCC's Dulbecco's modified Eagle medium (with 4 mM L-glutamine adjusted to contain 1.5 g/l sodium bicarbonate, 4.5 g/L glucose and 1 mM sodium pyruvate) and 10% fetal bovine serum.

Particles: Particles were tested from Bangs Labs, Spherotech, Dynal, and Polysciences. Concentration of particles can vary from vendor to vendor and lot to lot. Assay particle concentrations are optimized for a given lot by determining what concentration of particles provides the desired linear range. Particle concentration of beads can vary depending on fines in the bead suspension.
Detector moieties: Detector moieties from Jackson Immunochemicals, Chemicon, Caltag, and Ebiosciences were tested in the various assays. The best detector tested for a given assay format was used for further studies, and was optimized to give the best signal to noise ratio. The utility of the labeled detector moieties was also found to vary from vendor to vendor and lot to lot, and a given lot should be optimized for the desired assay for best performance. The immunoglobulin assays were found to work with F(ab)2 fragments or with whole antibodies as detectors.
Calibration standards: Commercially available standards, used to generate calibration curves, can also vary from lot to lot. Standard concentrations are determined by absorbance and then the standards are appropriately diluted to provide the desired range of concentration standards.

### Methods:

Preparation for Assay: IgG Capture beads were diluted with Assay buffer to provide an appropriate total volume per well (typically 50 ul). Mouse IgG detector was diluted 50 fold with Assay buffer. Standards were prepared by diluting antibody standard stock provided by Guava® Technologies in hybridoma media containing FBS. Standards in the range of 40-1.25 μg/mL were generated using serial dilution for mouse IgG assays and standards in the range of 0.5-20 μg/mL were generated for the human IgG Assay.
Mouse IgG Bead Assay: 2 μL of standards was added to a 48 μL volume of capture bead suspension in a microplate well and the plate incubated with gentle shaking for 40 minutes. 25 μL of a detector solution containing fluorescently labeled secondary antibody was added to each well and the plate incubated for 60 minutes. 125 μL of a Analysis buffer was next added to the beads and the plate was analyzed immediately on the Guava® EasyCyte™.
Human IgG Bead Assay: 7.5 μL of standards was added to a 45 μL volume of capture bead suspension in a microplate well and the plate incubated with gentle shaking for 40 minutes. 25 μL of a detector solution containing fluorescently labeled secondary antibody was added to each well and the plate incubated for 60 minutes. 125 μL of Analysis buffer was next added to the beads and the plate was analyzed immediately on the Guava® EasyCyte™.
Compatibility of Assay in Different Media: IgG standards were prepared by serial dilution in respective media. The IgG particle assay was performed as described above using respective standards and analyzed on the Guava® EasyCyte™. Standard Hybridoma Media used was ATCC's Dulbeccos's modified Eagle medium with 4 mM L-glutamine adjusted to contain 1.5 g/l sodium bicarbonate, 4.5 g/L glucose and 1 mM sodium pyruvate 90%; fetal bovine serum 10%. Hybridoma SFM (Serum-free, low protein media) and PFHM II Protein Free Hybridoma Media was from Gibco Invitrogen.
High Capacity Bead based Assays: The assay format described above for IgG concentration determination may be used for moderate capacity or high capacity ranges by modulating detector concentrations.

For example, an assay linear range of 1-40 microgram/ml is obtained for mouse IgG quantitation when 0.5 microgram of detector is used per well. A linear range of 1-80 microgram/ml can be obtained when a detector concentration of 1 microgram is used per well. For example, moderate capacity assays and high capacity assay are d.

Particular reagents of interest for the exemplified embodiments include Protein G Biomagplus Beads from Polysciences. Goat-anti mouse IgG Fc (gamma) F(ab)2 fragment and goat-anti human IgG Fc(gamma) F(ab)2 fragment were obtained from Jackson Immunochemicals. The standards used in the assays were mouse IgG2a from R&D Systems, and human IgG from Jackson Immunochemicals.

Data Acquisition and Analysis: Data were acquired on a Guava® EasyCyte™ instrument equipped with a 488 nm laser using the Guava® ExpressPlus.Software. For acquisition of samples, settings were adjusted using blank bead samples and data acquired. The Mean Fluorescence Intensity (MFI) of the beads in the PM3 channel was used in plotting the standard curve. The volume being analyzed may be diluted in a buffer (e.g. containing PBS and Tween20) prior to data acquisition.

### Example 1

### Schematic Design of a Particle-Based Analyte Assay

Quantitative screening of antibodies secreted by hybridoma provides useful information in the selection of optimal clones particularly where high capacity production is important. Current existing assays for IgG quantitations are tedious with multiple steps, have limited sensitivity ranges and involve dilution of hybridoma supernatants before analysis. The Guava® microcapillary platform has been previously shown to be useful for the specificity screening of secreted antibodies. In this study we demonstrate the additional capabilities of the system and its application to bead-based quantitative analysis.

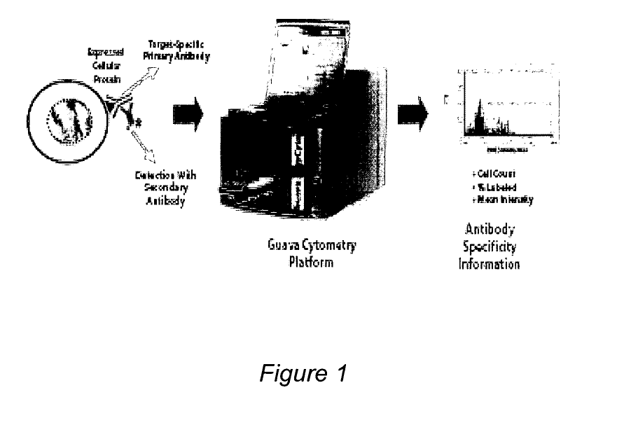

The Guava® platforms are microcapillary-based bench-top cytometry platforms that allow for manipulation and analysis of particles based on light scatter and fluorescence characteristics in either single tube or 96 well plate based formats, and can be used for performing the methods of the invention. A number of cell-based assays useful for the antibody production process have been developed on the platform such as the Guava® Express™ Assay which allows evaluation of specificity of generated antibodies for specific antigen of interest as shown in FIG. 1, ViaCount® for viability of cellular population, etc.

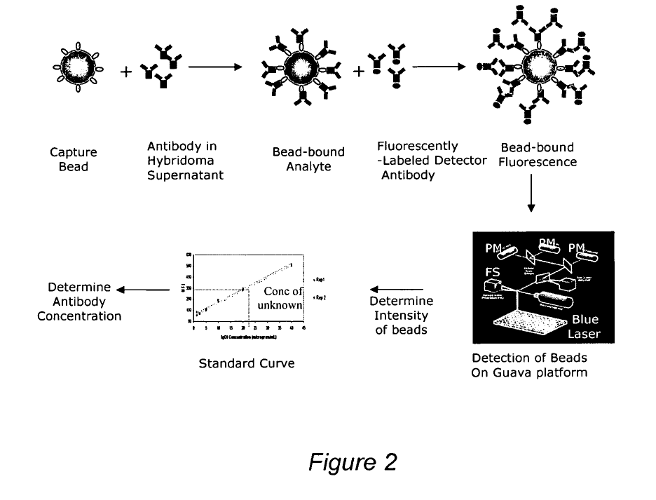

A quantitative particle-based assay to obtain concentration of secreted IgG is shown (FIG. 2). Hybridoma supernatant is directly incubated with polymeric particles coated with a capture reagent with affinity for Fc region of secreted antibody; the beads may be prepared or obtained commercially. Antibody in the supernatant binds to the particles and the particle-bound antibody analyte is detected with a fluorescently labeled secondary antibody. The entire mixture is then directly introduced into the Guava® cytometer and bead bound fluorescence is detected to determine Mean Fluorescence Intensity (MFI) from which the amount of antibody is calculated.

### Example 2

### Schematic Procedure for Particle-Based Characterization of Analytes in Hybridoma Supernatants

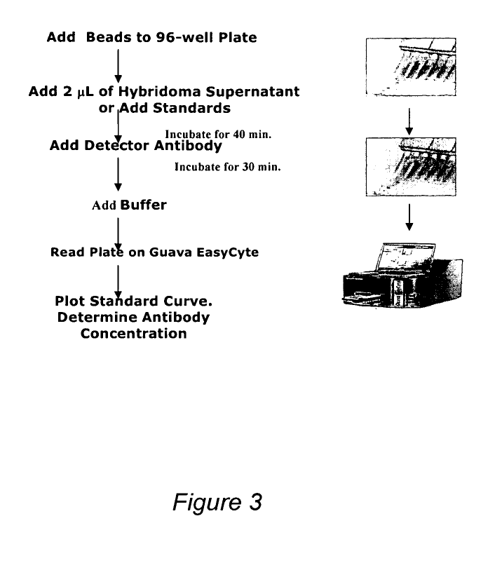

The Guava® Mouse IgG quantitation procedure is a simple mix-and-read procedure as shown in FIG. 3A. 2 μL of hybridoma supernatant is directly added to polymeric beads in microplates. After capture, a detector antibody is added and the mixture incubated. In the exemplified embodimens, the assay utilizes high capacity Biomag plus Protein G beads, which capture the secreted antibody in the hybridoma supernatant, followed by detection of bead-bound antibody using fluorescently labeled anti-mouse IgG detector antibodies. Buffer is added to the bead mixture, which is then immediately read on the Guava® EasyCyte™ system. The assay procedure allows for easy quantitation of antibody in supernatant without tedious dilution procedures or wash steps. The assay procedure is simpler, quicker and involves fewer hands-on steps compared to typical Elisa protocols for IgG quantitation as shown in FIG. 3B. Further no dilution of sample is needed if sample is from a typical hybridoma supernatant. Quantitation of bead-bound antibody is performed by analysis on the Guava® EasyCyte™ system. The bead-based assay demonstrates linear responses in the range of 1-40 microgram/mL and shows good responses for several mouse subtypes investigated. Standard curves may be generated and used to determine the concentrations of analytes.

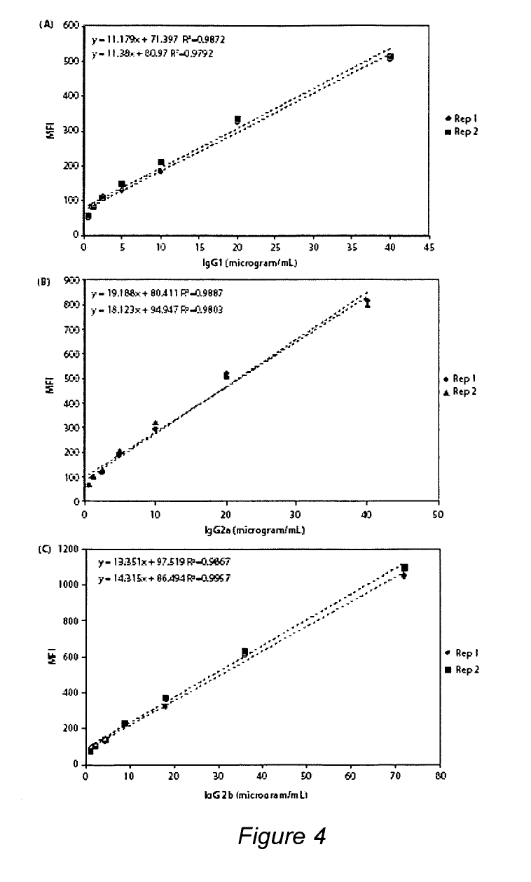

We examined the feasibility of performing a quantitative mouse IgG assay on the Guava® EasyCyte™ system. 2-5 μL of mouse antibody standards at different concentrations in standard hybridoma media with FBS were incubated with capture beads that have affinity for mouse antibodies. Bead-bound material was detected by using a secondary antibody with specificity for mouse heavy and light chains and analyzed on the Guava® EasyCyte™. The fluorescent response of the bead population for each concentration was plotted against the expected concentration (FIG. 4). The data demonstrate a linear response for each antibody isotype evaluated (mouse IgG1, IgG2a and IgG2b) in the range of 1-40 μg/mL as shown in FIG. 4A, B and C, with R2>0.98 in most cases. No recognition of mouse IgM antibodies was observed indicating the assay is specific for mouse IgG antibodies (data not shown). Thus, the bead-based total IgG assay can provide both specific identification of wells containing IgG antibodies as well as a quantitative measure of IgG levels in hybridoma media.

Identification and quantitation of antibody levels of a particular isotype is of value for people interested in screening for particular subtype as well as during optimization and production processes. In this example we developed and evaluated the performance of a bead-based mouse IgG1 assay. Mouse IgG1 antibody standards in standard hybridoma media with FBS were incubated with capture beads and the bead-bound material was detected with a fluorescently labeled IgG1 specific antibody followed by analysis as described under Methods. The quantitation showed excellent linear response in the 1-40 μg/mL region investigated (Panel A) with an R2=0.99. In addition the assay also demonstrated excellent specificity for IgG1 antibodies as shown in Panel B. No recognition above baseline was observed for equivalent amounts of mouse derived IgG2a, IgG2b, IgG3 or IgM antibodies analyzed (each at a concentration of 20 μg/mL) demonstrating the specificity of the assay for mouse IgG1 antibodies. The IgG1 quantitative assay thus can provide specific identification of wells containing mouse IgG1 antibodies and a quantitative measure of their levels.

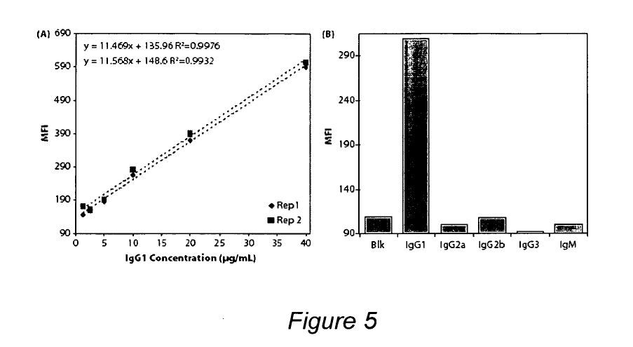

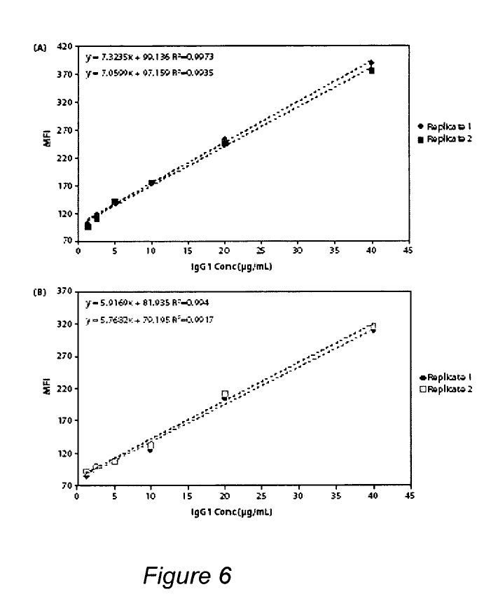

Hybridoma media used for antibody production can range from media containing FBS, to serum-free or protein-free media some of which can potentially contain interfering substances such as phenol red. We investigated the linearity of the IgG1 standards in three different media types—standard media which contains FBS as shown in FIG. 5A, serum-free low protein media as shown in FIG. 6A and serum-free, protein-free media as shown in 6B. In all three cases, quantitative linear responses were observed in the 1-40 μg/mL range of interest for the assay (R2=0.99 in all cases). The fluorescent response appears slightly modulated in FIGS. 6A and 6B possibly due to the presence of phenol red in the protein-free media. The data demonstrate that the assay is compatible with different hybridoma media but best results are obtained when the assay standards are diluted in the same media as samples to be analyzed.

### Example 3

### Preparation of a Standard Curve using the Guava® Mouse IgG Titer Assay on the Guava® Microcapillary Platform

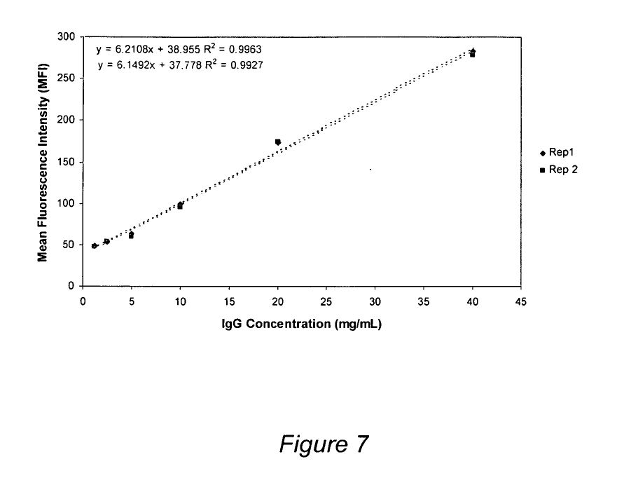

A typical standard curve obtained using the bead-based quantitation procedure described above is shown for the Mouse IgG Titer Assay (FIG. 7). The data demonstrate that this embodiment of the assay format provides excellent linear responses in the range of 2.5-40 μg/mL range using only 2 μL of hybridoma supernatant (R2=0.99). The standard curve generated can be used for prediction of IgG concentration of samples including hybridoma supernatants. No cross-recognition of mouse IgM antibodies was observed, indicating the assay is specific for mouse IgG antibodies (data not shown). Thus, the bead-based Mouse IgG Titer assay can provide both specific identification of hybridomas secreting IgG antibodies as well as a quantitative measure of their production levels.

### Example 4

### Performance of the Guava® Mouse IgG Assay in IgG Concentration Prediction

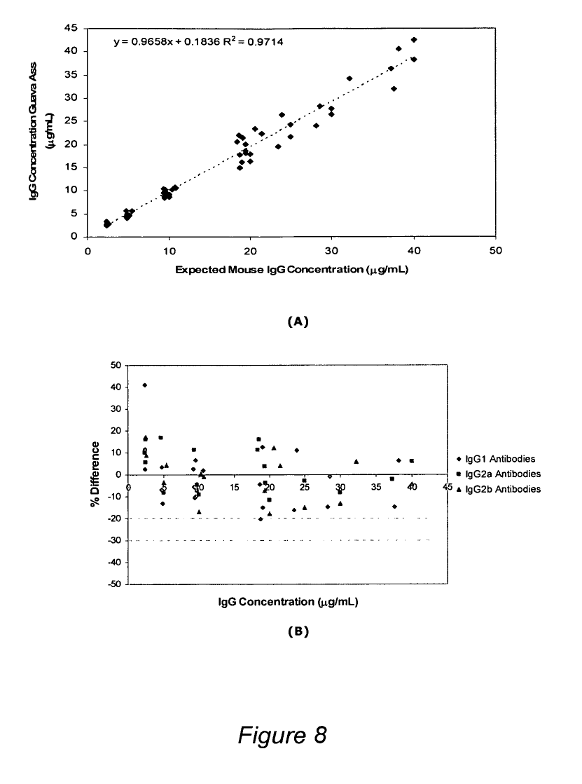

The accuracy of IgG concentration prediction was evaluated by using a number of standard antibodies whose concentration was determined by absorbance at 280 nm. Antibodies belonging to the IgG1, IgG2a and IgG2b subtypes were purchased from different vendors and their concentrations determined by absorbance. Fixed volume of the antibodies was diluted with hybridoma media so that they were in the linear range of the assay from ˜2.5 to 40 μg/mL. The concentration of these diluted solutions were next determined by the Guava® Mouse IgG Assay and the accuracy of the results compared. The plot in FIG. 8A demonstrates that excellent correlation can be obtained between the Guava® predicted concentration versus those determined by absorbance in the range of the assay. Further, the % Difference Plot (FIG. 8B) demonstrates that for the 12 antibodies tried at different concentrations an average % difference of ˜−0.49% was observed. The Guava® Mouse IgG kit thus provides accurate concentration prediction in the range of 2.5-40 μg/mL for different antibodies belonging to the IgG1, IgG2a and IgG2b subtypes and thus is a universal IgG quantitation kit.

### Example 5

### Precision of the Guava® Mouse IgG Assay in IgG Concentration Determination

The intra-assay precision of the assay was determined as described. Briefly, 6 replicates at three different concentration of mouse anti-human HLA-ABC antibody in hybridoma media containing FBS were tested and their concentration determined using the Guava® Mouse IgG Titer Assay. The CVs of the predicted IgG concentration are shown in each case. An average intra-assay of 5.3% was observed. The Guava® IgG Titer assay thus demonstrates good precision in antibody concentration prediction.

TABLE 1

| Concentration | Intra-Assay |
|---|---|
| Range (mg/ml) | Precision (% CV) |
| 20 | 4.6 |
| 10 | 4.9 |
| 2.5 | 6.5 |
| Average % CV | 5.3 |

### Example 6

### A High Capacity Mouse IgG Assay

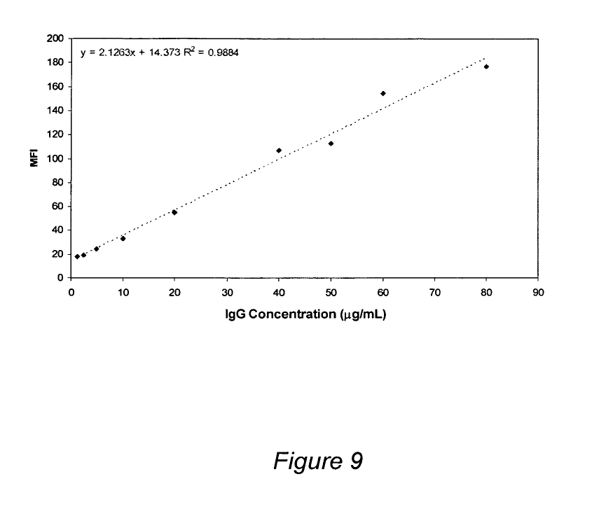

In several scenarios ranging from development to production antibody concentration ranges are in a much higher concentration range than what is encountered in hybridoma supernatants. Data from a High Concentration Range Mouse IgG Assay using a modified experimental format to the one described in the previous examples is shown (FIG. 9). Under modified conditions, a linear response (R2=0.98) for a much wider quantitative range of 1-80 microgram/mL could be obtained.

### Example 7

### A Particle-based Human IgG Titer Assay

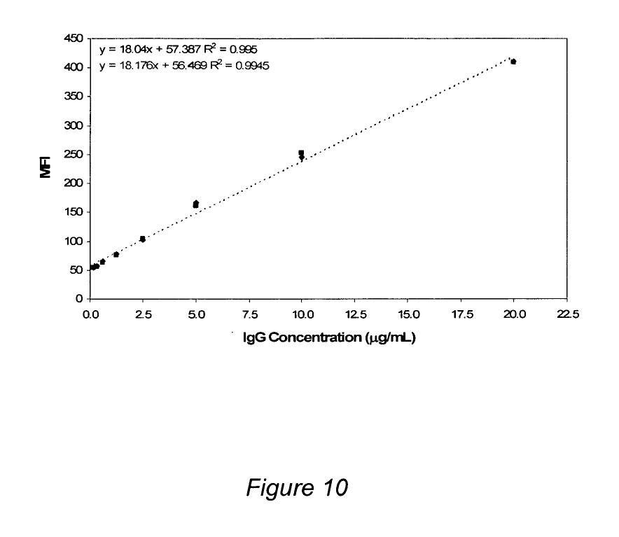

The Guava® particle-based assay approach can be utilized to create a number of other quantitative assays. In this example, data from a novel particle-based assay for determination of human IgG concentration is provided (FIG. 10). A representative standard curve demonstrates that the assay format in this embodiment provides excellent linear response in the range of 0.5-20 μg/mL using only 7.5 μL of supernatant. The assay is specific for human IgG and does not cross-recognize IgM antibodies. The assay can provide specific identification of IgG containing wells and quantitation of the antibody present. The assay can provide quantitation of all human IgG subtypes (IgG1, IgG2, IgG3 and IgG4). Both kappa and lambda chain antibodies can be quantitated using this approach.

### Example 8

### Performance of a Human IgG Titer Assay in Antibody Concentration Determination

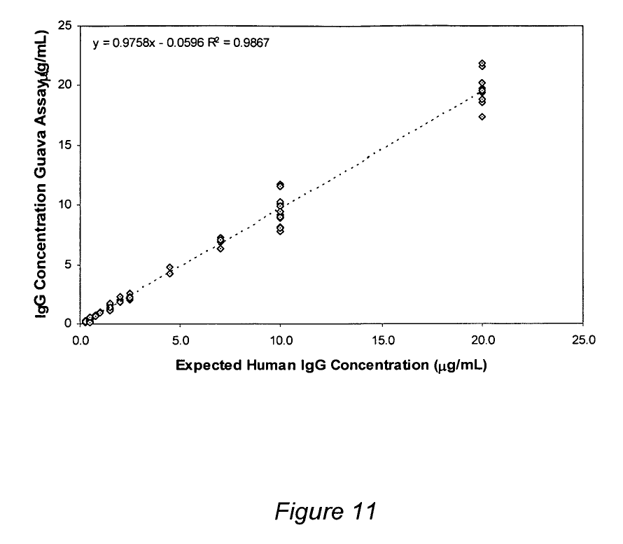

The accuracy of concentration prediction of the assay was evaluated by using a number of commercial antibodies whose concentration was determined by absorbance at A280. Antibodies belonging to the IgG1, IgG2, IgG3 and IgG4 subtypes were purchased and their concentrations determined by absorbance. Fixed volumes of the antibodies were diluted with hybridoma media so they were in the linear range of this assay embodiment (from ˜0.5 to 20 μg/mL). The concentration of these diluted solutions were next determined using the Guava® Human IgG Titer Assay and the accuracy of the results compared. The plot in FIG. 11 demonstrates that excellent correlation could be obtained between the predicted concentration using the particle-based assay as compared to that determined by absorbance over the entire tested range of the assay. The Guava® Human IgG Assay thus can provide accurate concentration prediction in the tested range of 0.5-20 microgram/mL for different antibodies belonging to the IgG1, IgG2 and IgG3 and IgG4 subtypes. Variations of assay parameters as described herein can permit optimization of this and other described assays where different concentration ranges are desired.

### Example 9

### Preparation and Use of a Kit for Analyzing Total Human IgG

A kit for detecting and/or quantitating total human IgG in a sample is prepared as described below, and an assay procedure for using the kit is also described. Instructions for performing the assay may be provided with the kit.

Preparation of Beads: 50 microliters of BiomagPlus Protein G beads from Polysciences is diluted in a buffer (PBS, 0.2% BSA, 008% sodium azide) to 5 mL. 42.5 uL of the bead solution is used in each well for capturing antibody for the human IgG assay.
Preparation of Anti-Human IgG Detector: Fluorescein (FITC)-conjugated AffiniPure F(ab′)2 Fragment Goat Anti-Human IgG, Fcγ Fragment Specific (minimal cross-reaction to Bovine, Mouse, and Rabbit Serum Proteins) (Jackson Immunochemicals) is diluted according to manufacturer's instructions. An equal volume of glycerol was added to the antibody and aliquots are frozen and provided with the kit. For use in the assay, a Working Solution of Anti-Human IgG Detector is prepared: 200 uL of antibody is diluted 12.5 fold to a total volume of 2500 uL using a buffer (PBS, BSA, 0.08% sodium azide).
Preparation of Standards for Assay: Human IgG Standard (Jackson Immunochemicals) is supplied at a concentration of 11 mg/mL and is diluted with buffer (PBS+0.08% azide). The standard is diluted to produce a 1 mg/mL standard which is supplied with the kit. This standard is further diluted with standard hybridoma media (or the particular media being tested) to produce standards concentrations from 20 ug/mL-0.313 ug/mL which are used in the assay.
Human IgG Bead Assay: 7.5 uL of each standard is added to a 42.5 uL volume of capture bead suspension in a microplate well and the plate is incubated with gentle shaking for 40 minutes. 25 uL of a Working Solution of Anti-Human IgG detector is prepared as described above is added per well, and the plate incubated for 60 minutes. 125 uL of Analysis Buffer (PBS, 0.05% Tween-20) is added to the beads and the plate is analyzed immediately on the Guava® EasyCyte™.

The kit can comprise beads, the detector, and optionally the buffer solution for preparing with Working Solution, the Analysis Buffer, and/or the assay standards.

### Example 10

### Preparation and Use of a Kit for Analyzing Total Mouse IgG

A kit for detecting and/or quantitating total mouse IgG in a sample is prepared as described below, and an assay procedure for using the kit is also described. Instructions for performing the assay may be provided with the kit.

Preparation of Beads: 50 uL of BiomagPlus Protein G beads from Polysciences are diluted in a buffer (PBS, 0.2% BSA, 008% sodium azide) to 5 mL. 48 uL of the bead solution is used in each well for capturing antibody for the mouse IgG assay.
Preparation of Anti-Mouse IgG Detector: Fluorescein (FITC)-conjugated AffiniPure F(ab′)2 Fragment Goat Anti-Mouse IgG, Fc-gamma Fragment Specific (minimal cross-reaction to Human, Bovine, and Horse Serum Proteins) is obtained from Jackson Immunochemicals and resuspended according to manufacturer's instructions. An equal volume of glycerol is added to the antibody and aliquots are frozen. For use in the assay, a Working Solution of Anti-Mouse IgG Detector was prepared by diluting 100 uL of Goat Anti-Mouse IgG antibody was diluted 25 fold to a total volume of 2500 uL using a buffer (PBS, BSA, 0.08% sodium azide).
Mouse IgG standard: Mouse IgG2a Standard (R&D systems) is diluted with buffer to make a 500 microgram/ml solution; the concentration is determined by absorbance and to diluted to provide a standard at a concentration of 200 ug/mL which is supplied with the kit. For the assay, the supplied standard at 200 ug/mL is diluted with hybridoma media (or other media used in the assay) to produce standards at 40 ug/mL-0.625 ug/mL and used in the assay to generate a standard curve.
Mouse IgG Bead Assay: 2 uL of each standard is added to a 48 uL volume of capture bead suspension in a microplate well and the plate incubated with gentle shaking for 40 minutes. 25 uL of a Working Solution of Anti-Mouse IgG Detector prepared as described above for the assay was added to each well and the plate incubated for 60 minutes. 125 uL of a Analysis buffer was next added to the beads and the plate was analyzed immediately on the Guava® EasyCyte™.

The kit can comprise beads, the detector, and optionally the buffer solution for preparing with Working Solution, the Assay Buffer, and/or the assay standards.

### REFERENCES

1. A fast and simple dot-immunobinding assay for quantification of mouse immunoglobulins in hybridoma culture supernatants. Sulimenko T, Draber P., J Immunol Methods. 2004, 289, 89-95.
2. Quantitation of monoclonal antibodies by ELISA. The use of purified mouse IgG and mouse IgM monoclonal antibodies as standards in a quantitative ELISA measuring monoclonal antibodies produced by cell culture. Mushens R E, Guest A R, Scott M L, J Immunol Methods. 1993, 162(1):77-83.
3. Indirect double sandwich ELISA for the specific and quantitative measurement of mouse IgM, IgA and IgG subclasses. Klein-Schneegans A S, Gaveriaux C, Fonteneau P, Loor F. J Immunol Methods. 1989, 119(1), 117-25.
4. Quantitative ELISA for mouse monoclonal antibody determination in culture supernatants and in human serum. Vilaseca J C, Pupo M, Bernal M, Matamoros L, Gordillo S, Rodriguez H, Caballero Y, Otero A. Hybridoma. 1997, 557-62.
5. Method for quantitation of IgG subclass antibodies in mouse serum by enzyme-linked immunosorbent assay. Gupta R K, Siber G R J Immunol Methods. 1995, 181(1):75-81.
6. Roback J D, Barclay S, Hillyer C D. Improved method for fluorescence cytometric immunohematology testing. Transfusion. 2004; 44(2):187-96.
7. Guava® CellToxicity Assay: A Novel Fluorescent Assay for Measuring NK Effector Function Katherine Gillis and Dianne M. Fishwild, Guava® Technologies Application Note, 2004.
8. A technology for the rapid acquisition of cell number and viability. Phi-Wilson J, Harvey J, Goix P, and O'Neill R. American Biotechnology Laboratory, May 2001. p 34-36.
9. Microsphere-based molecular cytometry. Iannone Mass. Clin Lab Med. 2001, 21(4):731-42.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

1-100. (canceled)

101. A method for analyzing a sample comprising:
providing a sample suspected of comprising a first analyte, said sample comprising a fluid medium;
providing a first particle comprising a first capture molecule for the first analyte, said first particle having a non-uniform shape;
providing a first detection moiety comprising a first label and a first binding means that localizes the first detection moiety to the first particle when the first analyte is bound to the capture molecule;
creating a test sample by combining the first particle, the sample and the first detection moiety in a solution;
analyzing the test sample or a portion thereof for an optical emission associated with the first label; and
determining the presence and/or concentration of the analyte in the sample.

102. The method of claim 101, wherein the first particle is smaller than 2 micron in size in one, two, three or all dimensions.

103. The method of claim 101 wherein the first particle comprises a material that is magnetic, paramagnetic or superparamagnetic.

104. The method of claim 101, comprising withdrawing a test volume of fluid comprising the first particle;
subjecting the test volume or a portion thereof to an excitation source; and
analyzing the test volume or portion thereof for fluorescence emission.

105. The method of claim 104, further comprising assaying the test volume or portion thereof and/or the test sample or portion thereof for at least one scatter parameter.

106. The method of claim 105, wherein the test volume or portion thereof and/or the test sample or portion thereof is assayed for forward scatter.

107. The method of claim 104, wherein the test volume is analyzed using an optical imaging system.

108. The method of claim 104, wherein the test volume is automatically withdrawn into a capillary at a uniform flow rate.

109. The method of claim 101, wherein the first analyte is an antibody, a fragment thereof, or a modified form of either thereof.

110. The method of claim 101, wherein the first binding means comprises an antibody-binding substance that binds to at least a fragment of an antibody.

111. The method of claim 110, wherein the antibody-binding substance binds to a plurality of different isotypes of antibodies, and wherein the first detection moiety can be used to quantitate the plurality of different isotypes if present in the sample.

112. The method of claim 110, wherein the antibody-binding substance binds to a plurality of different isotypes of antibodies, and wherein the first detection moiety binds to the Fc-gamma region of the antibodies and can be used to equivalently quantitate any of the plurality of different isotypes if present in the sample.

113. The method of claim 110, wherein the antibody-binding substance is specific for an isotype of an antibody and the first detection moiety can be used to determine the isotype of an antibody if present in the sample.

114. The method of claim 101, wherein the first binding means comprises an antibody-binding substance that can bind to a plurality of different isotypes of antibodies, and wherein the first detection moiety is used to quantitate an antibody if present in the sample;
further comprising contacting one or more aliquots of the same sample with one or more different isotype detection moieties specific for different antibody isotypes, each of said different isotype detection moieties comprising a label, wherein the one or more aliquots of sample are also contacted with isotype capture particles that comprise capture molecules that can bind to the antibody isotypes, and determining the isotype of an antibody if present.

115. The method of claim 101, wherein the sample further is suspected of comprising a population of cells.

116. A method for analyzing a sample comprising:
providing a sample suspected of comprising a first analyte and further suspected of comprising a population of cells, said sample comprising a fluid medium, said cells suspected of comprising a cellular detection moiety;
providing a first particle comprising a first capture molecule for the first analyte, wherein said first particle has a non-uniform shape and is optically distinguishable from the cell population;
providing a first detection moiety comprising a first label and a first binding means that localizes the first detection moiety to the first particle when the first analyte is bound to the capture molecule;
providing a second detection moiety comprising a second label and a second binding means that localizes the second detection moiety to the cellular detection moiety when present, wherein the first and second labels are optically distinguishable labels;
contacting the first particle with the sample and with the first detection moiety, and contacting the second detection moiety with the sample or a component thereof suspected of comprising the population of cells;
withdrawing a test volume of fluid suspected of comprising the first particle and/or a cell from said population;
analyzing the test volume for at least one optical emission associated with the first and/or second label;
analyzing the test volume for scatter; and
determining the presence and/or concentration of the analyte in the sample.

117. A method for analyzing a sample comprising:
providing a sample suspected of comprising a first analyte, said sample comprising a fluid medium;
providing a first particle comprising a first capture molecule for the first analyte;
providing a second particle comprising a second capture molecule for a second substance, wherein the second particle is optically distinguishable from the first particle, wherein the second substance interferes with the assay for the first analyte, said second particle having a non-uniform shape, and the second particle is used to reduce the amount of the second substance dissolved in the sample and thereby reduce its interference with the assay for the first analyte;
providing a first detection moiety comprising a first label and a first binding means that localizes the first detection moiety to the first particle when the first analyte is bound to the capture molecule;
contacting the sample with the first particle and with the second particle;
combining the first particle with the first detection moiety; and
determining whether the first label is associated with the first particle.

118. The method of claim 117, wherein the second substance is selected from immunoglobulins, albumin, adult red blood cells, fetal red blood cells, adult white blood cells, and fetal white blood cells.

119. The method of claim 101, wherein at least part of the assay is performed cytometrically.

120. The method of claim 101, wherein the first particle has at least one dimension of less than 2 microns and a non-uniform shape and a higher binding capacity for the first analyte as compared to a spherical particle of the same volume.

121. The method of claim 101, wherein the sample is a culture medium from a protein-secreting eukaryotic cell and the sample is assayed without prior dilution, wherein the sample volume assayed is from 0.5 nL-2 mL.

122. The method of claim 101, wherein assay standards at different concentrations are used to generate a calibration curve for the first analyte.

123. A kit for cytometric analysis of a sample comprising:
a first vessel containing a population of first particles, each of said population comprising a plurality of first capture molecules for a first analyte and having at least one dimension of less than 2 microns;
a second vessel containing a plurality of first detection moieties, each of said plurality comprising an optically detectable first label and a first binding means capable of localizing to the first particle when the first analyte is bound to the capture molecule; and
(a) a housing for retaining the vessels, or
(b) instructions for use of the components of the kit, or
(c) both (a) and (b).

124. The kit of claims 123, wherein the kit comprises a plurality of vessels each containing a different plurality of detection moieties, each of said different plurality of detection moieties comprising binding means specific for a different analyte.
